(19) United States
(12) Patent Application Publication
(10) Pub. No.: US 2006/0015018 A1
(43) Pub. Date: Jan. 19, 2006

(54) CAS modular body reference and limb position measurement system

(76) Inventors: Sebastien Jutras, Montreal (CA); Herbert Andre Jansen, Montreal (CA); Benoit Chouinard, Montreal (CA); Andrew G. Yun, Inglewood, CA (US); Isabelle Fontaine, Montreal (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/196,754

(22) Filed: Aug. 4, 2005

Related U.S. Application Data
(63) Continuation of application No. PCT/CA04/00159, filed on Feb. 4, 2004.

(51) Int. Cl. A61B 5/00

(52) U.S. Cl. 600/301

(57) ABSTRACT

A surgical bone reference assembly for communication with a CAS system, comprises a bone anchor fastenable to a bone element, an adjustable support and a trackable member that is located and tracked in three dimensional space by the CAS system. The support is removably fastenable to the bone anchor member and permits variable positioning relative to the bone anchor member. The support is intra-operatively detachable from and re-fastenable to the bone anchor member. A method of using a CT-free CAS system for determining a change in position of an un-tracked target limb is also provided. The method comprises engaging the trackable bone reference member to another bone element, locating and digitizing a landmark on the target limb, digitizing the landmark again following joint reduction, and determining at least one of a post-joint reduction limb length discrepancy and a target limb medio-lateral offset.

### CROSS-REFERENCE TO RELATED APPLICATIONS

This is a continuation of International Application No. PCT/CA2004/000159 filed Feb. 4, 2004, designating the United States, which itself claims priority on U.S. provisional application 60/404,758 and 60/444,691 which were respectively filed Apr. 30, 2003 and Feb. 4, 2003, the specifications of all of which are incorporated herein by reference.

### TECHNICAL FIELD

The present invention relates generally to a trackable reference for use in conjunction with a Computer Assisted Surgery (CAS) system.

### BACKGROUND OF THE INVENTION

CAS systems capable of real time location and tracking of a plurality of CAS identifiable markers in a surgical field are desirable. A variety of systems are now employed, some of which require that the necessary bone elements of the patient be identified and registered to pre-operatively taken anatomical scans or intra-operatively taken images of the same bone elements. In order for the relevant bone elements to be located and tracked by the CAS system, trackable reference members must be fastened thereto. These bone reference members will vary depending on the type and specific requirements of the particular CAS system used. However, CAS surgical procedures have more recently tended towards systems and surgical methods which do not require such anatomical scans or images in order to identify the bone elements of the patient. As such procedures do not require CT scans to generate the pre-operative anatomical models, these procedures are often termed CT-free or CT-less operations.

No matter the CAS operation system employed, in order for the relevant bone elements to be located and tracked by the CAS system, trackable reference members are nevertheless typically used to identify the position and orientation in space of the bone element. These bone reference members vary depending on the type and specific requirements of the particular CAS system used.

For example, for an optical CAS system, the trackable bone reference members comprise at least three optically detectable markers whose exact positions can be determined by each of the at least two cameras of the optical CAS system. This therefore permits the position in space of each detectable marker to be determined by the CAS system, and therefore permits the position and orientation of the bone reference member, and consequently also the position and orientation of the bone element to which it is affixed, to be determinable by the CAS system.

For optically based CAS systems, the ability to maintain an unobstructed line of sight view between the system cameras and the detectable marker elements of the trackable member is of prime importance. This can, however, become difficult in some surgical installations, where numerous medical staff and a large quantity of medical equipment are required within the surgical field. The cameras of the CAS system must be able to simultaneously visually locate both the bone reference trackable member and any additional trackable members disposed on tracked tools employed. While tracked surgical instruments can more easily be displaced such that their trackable members are in an optimal position relative to the cameras, it is often more difficult and impractical to adjust the trackable bone reference member, being fastened to a bone element of the patient.

No matter what type of positioning reference block is used, all such reference members used in conjunction with a computer assisted surgery must comprise a trackable member. It is well known to permanently fix such trackable members to the reference block by such methods as welding, press-fitting, and pinning. However, as it can be desirable in particular circumstances to be able to separate the trackable member portion from the base reference block, it is known to fasten the trackable member to the reference block with releasable engagement mechanisms. These generally permit the trackable member to be completely removed from the reference member fastened to the bone element. This can be useful if temporary removal of the trackable member provides better access for the surgeon to a particular location, for example as may be helpful in hip surgeries.

However, once the trackable member is removed from the reference block fixed to the bone element, the position and orientation of the bone element is no longer known. As such, when the trackable member is re-attached to the reference member in an alternate position, the bone element must be re-registered in order for the CAS model or image to correspond to the position and orientation of the actual bone element, such that the reference member can then be again used to accurately track the bone element to which it is fixed.

Therefore, while the ability to remove a trackable member from a bone reference and re-engage it therewith intra-operatively is desirable, the re-registration that is subsequently required is time consuming and impractical. Additionally, known bone reference members provide limited adjustability of the trackable member. Maintaining an optimal, unobstructed visual contact between the bone reference trackable member and the cameras of the CAS system is consequently often difficult.

Hip surgeries in general, and total hip replacements in particular, are common. When total hip replacements are performed, there can be a discrepancy between the leg length on the treated hip side relative to the length of the non-treated leg. Additionally, replacement of the natural hip with a prosthetic replacement can also result in a change in the position of the leg of the treated hip along the medio-lateral axis of the pelvic coordinate system. However, any post-operative change in the longitudinal and medio-lateral positioning of the limb relative to the pre-operative values of the natural hip cannot easily be determined unless a trackable bone reference member is fastened to the limb in question.

### SUMMARY OF THE INVENTION

It is accordingly an object of the present invention to provide an improved CAS bone reference assembly having a trackable member adapted for communication with an image guided surgical system.

It is another object of the present invention to provide a CAS bone reference assembly having a trackable member that is selectively removable from a base reference member to which it is engaged, and re-engageable intra-operatively.

It is another object of the present invention to provide a CAS bone reference assembly comprising a selectively disengageable articulated support for a trackable member.

It is also an object of the present invention to provide a CT-free CAS system capable of determining limb position change relative to a pre-operative position of the limb.

It is another object of the present invention to provide a method for determining length discrepancy and medio-lateral offset of an un-tracked limb using a CT-free CAS system.

The present invention is generally directed to a bone reference having a selectively removable articulated support for a position identifying element trackable by a CAS system, and a method for determining the limb length discrepancy and limb medio-lateral offset in a computed tomography (CT) free total hip replacement surgery using the bone reference and the CAS system.

Therefore, in accordance with the present invention, there is provided a surgical bone reference assembly, adapted for communication with a computer assisted surgical system, comprising: a bone anchor member, engageable to a bone element of a patient such that substantially no relative movement therebetween is possible; a trackable member comprising a detectable element adapted to be located and tracked in three dimensional space by the computer assisted surgical system, thereby defining position and movement of said trackable member; an adjustable support member having said trackable member disposed at a first end thereof, a second end of said support member being removably fastenable to said bone anchor member by an attachment member, said support member permitting variable positioning of said trackable member relative to said bone anchor member and being lockable to fix said trackable member in a desired position relative to said bone anchor member; and said support member, via said attachment member, being intra-operatively detachable from said bone anchor member and subsequently re-fastenable thereto such that said trackable member is in said desired position and orientation relative to the bone element.

There is also provided, in accordance with the present invention, a method for monitoring position and movement of a bone element using a computer assisted surgical system comprising: fastening a bone anchor member to the bone element; attaching an adjustable support member to said bone anchor member, said adjustable support member having a trackable member fixed thereto, said trackable member including a detectable element being locatable and trackable in three dimensional space by said computer-assisted surgical system; adjusting said trackable member into a desired position and orientation relative to sensing elements of said computer-assisted surgical system; locking said adjustable support member in place such that said trackable member is fixed in said desired position and orientation relative to sensing elements of said computer-assisted surgical system; performing a registration of the bone element; detaching said adjustable support member from said bone anchor member; and re-fastening said adjustable support member to said bone anchor member, said trackable member being in said desired position and orientation without requiring readjustment and said bone element being locatable and trackable using said computer assisted surgical system without requiring re-registration of said bone element.

There is further provided, in accordance with the present invention, a method of using a computed tomography (CT) free computer assisted surgery (CAS) system for determining a change in position of an un-tracked target limb undergoing orthopaedic surgery, the method comprising: engaging a bone reference member, trackable by said CAS system, to a bone element distinct from said target limb, and using said bone reference member to define a base coordinate system; locating a position identifying landmark on said target limb; performing a first digitization of said landmark; performing a second digitization of said landmark following joint reduction; and determining at least one of a post-joint reduction limb length discrepancy value and a target limb medio-lateral offset value.

There is also provided, in accordance with the present invention, a computed tomography (CT) free computer assisted surgery (CAS) system for determining a change in position of an un-tracked target limb undergoing orthopaedic surgery, comprising: a bone reference member trackable by said CAS system and engaged with a bone element distinct from said target limb; means for locating said bone reference member and determining a base coordinate system relative thereto; a digitizer, trackable by said CAS system, for performing a first and a second digitization of a landmark on said target limb; means for determining pre-joint dislocation coordinates in said base coordinate system from said first digitization and post-joint reduction coordinates in said base coordinate system from said second digitization, and for determining longitudinal axis components and medio-lateral axis components of said pre-joint dislocation coordinates and said post-joint reduction coordinates; and means for determining at least one of a post-joint reduction limb length discrepancy value and a target limb medio-lateral offset value, said post-joint reduction limb length discrepancy value being computed using said longitudinal axis components and said target limb medio-lateral offset value being computed using said medio-lateral axis components.

There is finally provided, in accordance with the present invention, a method of using a computed tomography (CT) free computer assisted surgery (CAS) system for determining a change in position of a target limb undergoing orthopaedic surgery, the method comprising: engaging a first bone reference member, trackable by said CAS system, to a bone element distinct from said target limb, and using said bone reference member to define a base coordinate system; engaging a second bone reference member, trackable by said CAS system, to said target limb; performing a first digitization of said second bone reference member to identify a pre-joint dislocation position thereof relative to said base coordinate system; performing a second digitization of said second bone reference member, following joint reduction, to determine a post-joint reduction position thereof relative to said base coordinate system; and determining at least one of a post-joint reduction limb length discrepancy value and a target limb medio-lateral offset value.

### BRIEF DESCRIPTION OF THE DRAWINGS

Further features and advantages of the present invention will become apparent from the following detailed description, taken in combination with the appended drawings, in which:

FIG. 1 is a front perspective view of a surgical bone reference assembly according to the present invention;

FIG. 2 is a front perspective view of a disengageable trackable member portion of the surgical bone reference assembly of FIG. 1;

FIG. 3 is a front perspective view of a bone reference base portion of the surgical bone reference assembly of FIG. 1; and

FIG. 4 is a flow chart of a method of limb length discrepancy and medialization value determination according to a preferred embodiment of the present invention.

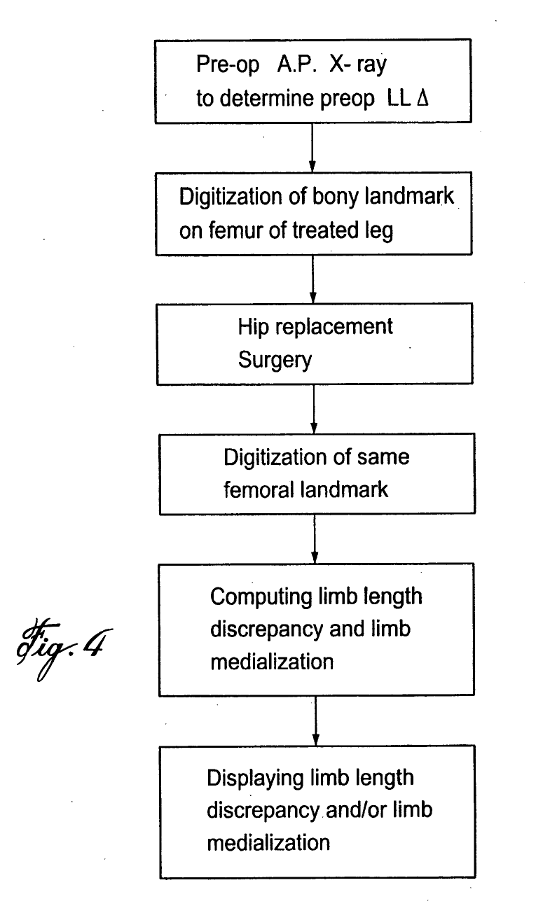

### DETAILED DESCRIPTION OF THE PREFERRED EMBODIMENT

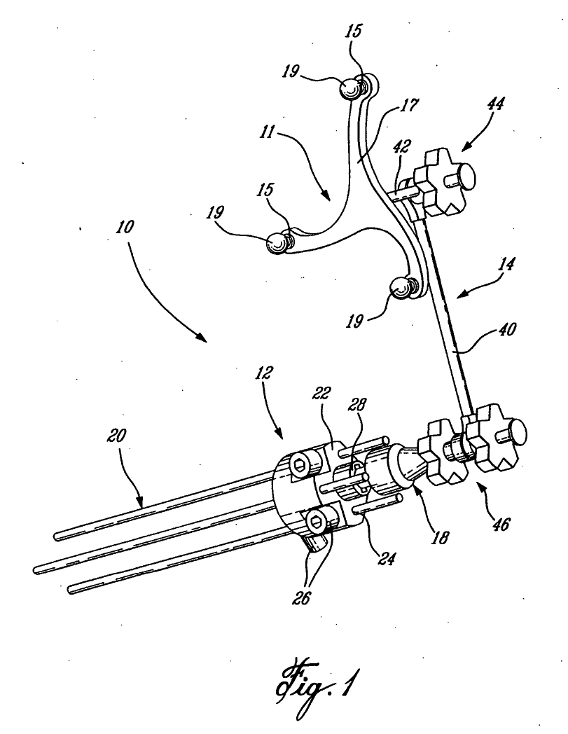

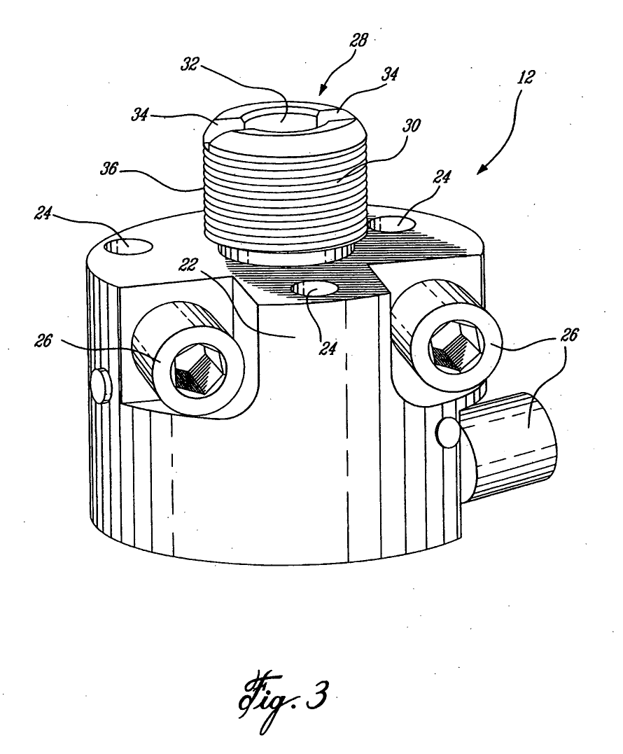

Referring to FIG. 1, a surgical bone reference assembly 10 generally comprises a bone anchor member 12, an articulated tracker support 14, having a trackable member 16 engaged at one end thereof, and being removably engageable and disengageable with the bone anchor member 12 by an attachment member 18. The trackable member 16 is adapted to be communicable with a computer assisted surgery (CAS) system capable of detecting and tracking the device in three-dimensional space within a surgical field. As best seen in FIG. 3, the bone anchor member 12 comprises preferably a cylindrical body 22 having at least one pin hole 24 extending axially therethrough for receiving at least one bone mounting pin 20 (FIG. 1). Preferably, however, three bone mounting pins 20 are used to fasten the bone anchor block 12 to a bone element of a patient such that no movement of the bone anchor member 12 relative to the bone element is possible. Three pin holes 24 are accordingly provided in the cylindrical body 22 of the bone anchor member 12, within which the bone mounting pins are received. Locking screws 26 extend transversely through the cylindrical body 22, such that their hidden tips can frictionally engage the bone mounting pins 20 disposed within the pin holes 24. The locking screws 26 permit the cylindrical body 22 to be axially adjusted on the bone mounting pins 20 and engaged thereto such that the bone anchor member 12 is fixed in place on bone mounting pins 20. The bone mounting pins are fastened into a bone element of a patient, preferably such that they protrude sufficiently therefrom to permit exposed distal ends of the pins extend beyond the soft tissue surrounding the bone element. Consequently, once the bone mounting pins are fixed in place, the bone anchor member 12 can be engaged thereto super-cutaneously (i.e.: above the skin), thereby being fixed relative to the patient without being directly fastened thereto. This reduces the invasiveness of the installation of the bone anchor member 12.

The pin holes 24 are preferably parallel to one another, however they can also be slightly inclined relative to one another. This requires each of the bone mounting pins 20 to be anchored into the bone element at a corresponding angle. This alternate arrangement can be used to provide better stability of the anchor member 12 when engaged to the bone mounting pins 20.

Although three bone mounting pins 20 are preferably used to fasten the bone anchor member 12 to a bone element of a patient, it is also possible to engage the bone anchor member 12 to at least one bone mounting pin or rod. Such a bone mounting pin or rod having a non-circular cross-sectional area received into a correspondingly shaped aperture or bore in the bone anchor member 12, would similarly prevent the possibility of the reference assembly 10 from rotating relative to the bone element 11, and the anchor member 12 could similarly be axially fastened thereto. Although not as secure as the use of three bone mounting pins 20, the use of a single, non-circular bone mounting pin would substantially eliminate relative movement between the anchor member 12 and the bone element, while requiring only a single insertion point for mounting the bone reference assembly 10 to the bone element. The use of two or three pins with such non-circular cross-sectional area is also possible. Further, a single pin can alternately be used which permits engagement of a toothed body directly with the bone element, the toothed body being fastened to, or integrally formed with, the bone anchor member 12.

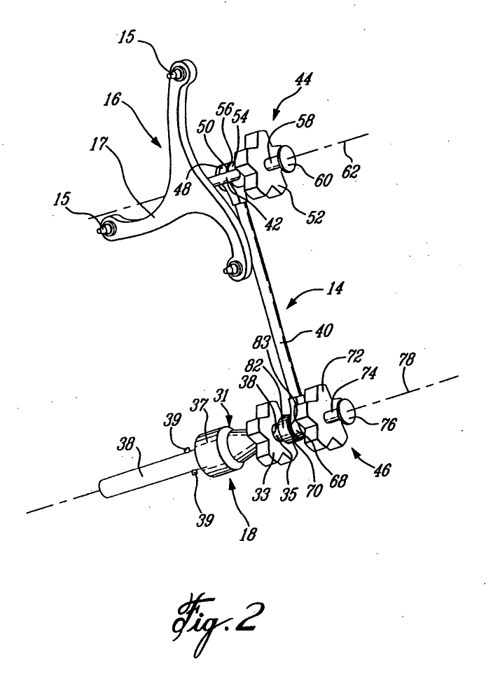

The bone anchor member further comprises a central mounting element 28, which is integrally formed with the cylindrical body 22 and distally extends therefrom. The outer circumferential surface 30 of the mounting element 28 preferably has external threads 36 thereon. A central bore 32 extends through both the mounting element 28 and the cylindrical body 22, and is sized to receive a proximal end of a base link member 38 (FIG. 2) therein. A transverse alignment groove 34 diametrically extends across the distal surface of the mounting element 28 on either side of the central bore 32. Each side of the alignment groove 34 receives transversely projecting alignment pins 39 of the base link member 38, as will be described in further detail below. The alignment groove 34 is preferably V-shaped, such that a corresponding pin, having a circular cross-sectional area, when disposed therein will always tend to be centrally located.

The trackable member 16 generally comprises a detectable tracker head element 17, including detectable element mounting posts 15 for receiving detectable markers thereon, which is connected to the bone anchor member 12 by an articulated support member 14 that will be described in further detail below. To each mounting post 15 is removably fixed a detectable marker element, such as an optically detectable sphere element 19. The detectable spheres 19 are preferably coated with a retro-reflective layer in order to be detected by, for example, an infrared sensor using axial illumination. Cameras of an optical CAS system can therefore detect the position of each optically detectable sphere 19 illuminated by infrared light. Each detectable marker element can equally be any other type of position indicator such as a light emitting diode or detectable electromagnetic indicator, provided each can be detected by the type of sensor used by the particular CAS system employed. Although the present surgical bone reference assembly 10 is preferably adapted for use with an optically based CAS system, one skilled in the art will appreciate that in addition to the optical and electromagnetic systems mentioned above, other types of CAS systems can also be used, such as, for example, those which use ultrasound or laser as a means for position identification. In such cases, it is to be understood that the detectable sphere elements 19 will be such that they are able to indicate to, or be detected by, sensors of the particular CAS position identification system used.

The articulated support 14 adjustably links the trackable member 16 to the anchor member 12. The articulated support 14 permits selective adjustability of the position in space of the trackable member 16 relative to the bone anchor member 12, and therefore to the bone element to which the bone anchor member 12 is fixed. The articulated support member 14 preferably comprises at least two independently articulated joint assemblies, such as first and second joint assemblies 44 and 46 in FIGS. 1 and 2. However, a single joint is equally possible. For example, a single rotating joint can be used between the bone anchor member 12 and an angled, rigid support arm having a trackable member on the end thereof. Although providing less adjustability and range of motion, such an arrangement would be simpler and less expensive. No matter the number, each joint preferably provides an independent single degree of freedom. However, a selectively lockable, ball-and-socket type joint could also be used, and would provide itself three rotational degrees of freedom. While joints providing rotational movement are preferred, other types of joints, for example those providing a translational degree of freedom, are equally possible, but preferably used in combination with at least one rotational joint.

Referring to the preferred embodiment as depicted in FIGS. 1 and 2, the articulated support member 14 comprises a first link member 40 and a second link member 42, interconnected by the first joint assembly 44 therebetween. The second link member 42 comprises a rigid rod element, fixed at one end to the tracker head element 17 of the trackable member 16, and having a preferably integrally formed annular second link end 54 at an opposing end. The annular second link end 54 includes a serrated, or toothed ring 56, disposed substantially perpendicularly to the surface of the tracker head element 17. The toothed rings are all preferably integrally formed with their annular link ends, however the toothed rings can also be separately formed and press fit, or otherwise securely engaged, with the link ends. The serrations or teeth of the toothed ring 56 inter-engage with corresponding teeth of a toothed ring 50, preferably integrally formed on an annular first link end 48 of the first link member 40. When the two toothed rings 50 and 56 are pressed into engagement together, the teeth interlock to prevent rotational movement relative to one another.

The annular first link end 48 comprises a central aperture defined therethrough, about which the toothed ring 50 disposed. The central aperture in the distal first link end 48 is concentric with a first joint axis of rotation 62, substantially perpendicular to a longitudinal axis of the first link member 40. A first axle pin 58 is permanently fixed at one end to the second link end 54, and extends through the central aperture in the annular first link end 48. The first joint axle pin 58 has an externally threaded central portion, not seen in the figures but disposed generally partially beneath each of a first joint locking nut 52 and the annular first link end 48. The central aperture through the first link end 48 has a diameter sufficiently large enough such that the axle pin 58 is free to rotate within the aperture. The axle pin 58 also comprises a disc flange 60 at the free end of the pin 58 opposed to the end fixed to the second link end 54. The disc flange 60 prevents the first joint locking nut 52 from being completely separable from the first joint assembly 44. When the locking nut 52, having internal threads corresponding to those on the axle pin 58, is tightened, it forces the annular first link end 48 towards the second link end 54, such that the corresponding toothed rings 50 and 56 engage one another. This thereby engages the first and second link members 40 and 42 in a specific angular relation to one another. The first joint assembly 44 therefore permits selective rotational adjustment of the second link member 42, to which the trackable member 16 is fastened, about the first axis of rotation 62.

The articulated support 14 further comprises a second joint assembly 46, providing selective rotational adjustment between the first link member 40 and the base link member 38 about a second joint axis of rotation 78, collinear with a longitudinal axis of both the base link member 38 and the bone anchor member 12. The second joint assembly 46 operates much as the first joint assembly 44, permitting selective rotation of the first link member 40 relative to the base link member 38 when a second joint locking nut 72 is disengaged, and fixed engagement between the base link member 38 and the first link member 40 when the second joint locking nut 72 is tightened. The second joint assembly 46 includes a proximal first link end 68, disposed at an opposite end of the first link member 40 from the distal first link end 48. The proximal first link end 68 comprises a toothed ring 70, having proximally projecting teeth for engagement with the distally projecting teeth of a corresponding toothed ring 83, centrally disposed on a distal base link end 82. A second joint axle pin 74 is fixed to the base link member 38 with the toothed ring 83, and distally extends therefrom. Much as the first joint axle pin 58, the second joint axle pin 74 has a threaded central body portion, such that the second joint locking nut 72 can be engaged thereto, thereby forcing the first link member 40 into fixed engagement with the base link member 38 when the locking nut 72 is tightened. The corresponding teeth of the mating toothed rings 70 and 83 on both the proximal end 68 of the first link member 40 and the distal end 82 of the base link member 38, are consequently engaged such that these two components are rotationally fixed relative to one another.

All surfaces of the present bone reference assembly 10 can be easily cleaned. Particularly, all surfaces of the joints can be sufficiently exposed such that thorough pressure cleaning is possible. The ability to sterilize all surfaces of the bone reference assembly 10 by pressure cleaning and autoclaving is important to ensure that all contaminating biological matter can be safely removed. Such potentially dangerous contaminating biological matter can include unwanted bacteria and proteins, which can cause infections or diseases. The free end flanges 60 and 76 of the joint axle pins 58 and 74 are spaced sufficiently away from the joints that the joint locking nuts 52 and 72 can be completely unscrewed and the two halves of the joints separated such that all surfaces, including the outer threads of the joint axle pins, can be substantially exposed to permit pressure cleaning thereof. Further, the articulated support member 14 is removable from the bone anchor member 12, as will be described below, which permits intra-operative sterilization of the articulated support member 14 and the trackable member 16 when required.

The articulated support member 14 is removably engageable to the bone anchor member 12 with an attachment member 18. The attachment member 18 comprises a main body 31 having a central bore 35 axially extending therethrough such that the main body 31 can be freely rotated on the base link member 38. The main body 31 includes a radially extending finger grip portion 33, integrally formed or permanently fixed to the main body 31, such that the main body 31 can be manually rotated. The bulbous proximal end 37 of the main body 31 has a greater outer diameter than the central portion of the main body 31, and comprises internal threads in the central bore 35 therewithin. The attachment member 18 thereby provides a tightening nut for screwed engagement with the bone anchor member 12. The internal threads of the nut portion of the main body 31 are co-operable and engageable with the external threads 36 on the circumferential outer surface 30 of the mounting element 28 of the bone anchor member 12, such that the proximal end 37 of the attachment member 18 can removably fasten the base link member 38 to the bone anchor member 12.

To fasten the articulated support member 14 to the bone anchor member 12, the proximal end of the base link member 38 is inserted into the central bore 32 of the bone anchor member 12, and transversely projecting alignment pins 39 which extend from the base link member 38 are aligned with, and inserted into, the alignment grooves 34 in the mounting element 28 of the bone anchor member 12. The alignment pins 39 thereby prevent unwanted rotation of the base link member 38, and consequently the entire articulated support member 14, relative to the fixed bone anchor member 12. The attachment member 18 can then be screwed into engagement with the mounting element 28 on the bone anchor member 12. Removal of the articulated support member 14 and trackable member 16 from the bone anchor member 12, is accordingly quickly and easily possible, by unscrewing the attachment member 18 from the mounting element 28, and axially sliding the base link member 38 out of the central bore 32 of the bone anchor member 12. The entire articulated support member 14, having the trackable member 16 disposed at an end thereof, can thereby be disengaged from the bone anchor member 12 which is fixed to the bone of the patient. This can be done intra-operatively, if for example, the patient has to be displaced or repositioned, and the articulated support member 14 would impede such required movement.

Similarly, the articulated support member 14 and trackable member 16 can thus be intra-operatively removed, sterilized and easily re-installed, without having to remove the bone anchor member 12, in substantially the exact same position and orientation relative to the bone element. As the bone anchor member 12 and the bone mounting pins 30 fixed to the bone element of the patient do not have to be removed, significant time savings can thus be made. The alignment pins 39 of the base link member 38 permit the articulated support member 14 to be re-positioned in the same orientation relative to the bone anchor member 12 when the trackable member 16 is to be re-engaged to the bone element. However, when the attachment member 18 is re-engaged with the bone anchor member 12, care must be taken to ensure that the articulated support member 14 is disposed in the same orientation as it was during the initial registration or digitization of the coordinate system, before the articulated support member 14 and trackable member 16 were disengaged from the bone anchor member 12, and is not replaced 180 degrees out of position.

In a preferred application, the surgical bone reference assembly 10 is used in a total hip replacement surgery, and is fixed to the ilium of the patient. Particularly, the surgical bone reference assembly 10 is preferably used in computer assisted hip surgery procedures, such as the CT-less THR surgery as described below, which do not use pre-operatively taken scans, such as computed tomography (CT) scans, to create a computerized bone model. Generally, the pelvic region of the patient is intra-operatively digitized to create a pelvic coordinate system. Although the actual hip replacement surgery is performed with the patient in a lateral decubitus position, the bone reference assembly 10 must be fixed to the ilium while the patient is in a supine decubitus position, to allow digitization of the pelvic coordinate system. Therefore, the articulated support member 14 and the trackable member 16 can be detached from the bone anchor member 12, which is fixed to the ilium by the bone mounting pins 20, once the digitization of the pelvic coordinate system is complete. As described above, this is done by unscrewing the attachment member 18. With the articulated support member 14 disengaged, the patient can then be displaced into the lateral decubitus position, without concern for the trackable member 16 and the associated articulated support structure. Additionally, the articulated support member 14 and the trackable member 16 can be sterilized if required once removed. Once the patient has been placed in the desired position for the surgical operation, the articulated support member 14 can subsequently be re-attached to the bone anchor member 12 in the same position it was in when the digitization was performed, and can be used to accurately locate and track the bone element without requiring a further registration or calibration of the trackable member 16 relative to the bone element.

As mentioned above, the CAS bone reference assembly 10 of the present invention is preferably intended to be used in conjunction with an optical tracking CAS system which employs a network of cameras to locate the trackable member 16, or more specifically to locate identification markers 19 of a detectable element 17 thereof, so that their position and movement can be tracked during the surgery. Therefore, when the bone reference assembly 10 is fixed to the desired patient bone element, such as the pelvic bone, the anatomical position and orientation of the bone element can be determined and tracked in space by the CAS system.

Although the present invention is preferably used with a CT-free CAS system, it is nevertheless to be understood that the step of performing a registration of the bone element, as used herein, comprises all means of relating the actual bone element to a corresponding model or image of the same bone element. Those skilled in the art will appreciate that there are a plurality of ways of creating such a model or image of the bone element, and of relating or matching the actual bone element to the model or image thereof.

When a CT-free surgical procedure is being used, once the bone reference assembly 10 is securely engaged to the bone element, thereby fixing the bone element relative to the location of the trackable member 16 of the bone reference assembly 10, a plurality of points on the relevant surfaces of the bone element can then be digitized to create a computer model of the surface. This is preferably done by acquiring the plurality of points, either pre-determined and sequentially identified by the CAS to the surgeon or randomly selected by the surgeon, on the surface of the bone element using a calibrated CAS probe.

Such landmark digitization techniques permit intra-operatively acquired surface points, preferably acquired on specific predetermined landmarks of the bone element surface, to be used to create the computerized anatomical reference model of the bone element. This eliminates the need for a CT scan, taken pre-operatively for example, to be used to generate the computer reference model of the bone element.

All methods of generating a computerized model or displaying image of the bone element, and of relating or matching the position and orientation of the actual bone element thereto, will be understood herein to be, included in the process of performing a registration of the bone element.

Generally, as will be described in greater detail below, once a landmark on the bone surface of the target limb is digitized, the CAS system can identify the position in the base coordinate system defined by the bone reference assembly 10 fixed to the bone element that is distinct from the target limb, such as the pelvic bone. The second digitization performed after the limb reduction similarly positions the target limb in the base coordinate system at this later time. These coordinates can then be used, as defined above, in order to determine the limb length discrepancy and the limb medio-lateral offset.

While another aspect of the present invention will be particularly described below with regard to a total hip replacement (THR) using a CT-free CAS system, it is to be understood that the present invention can be similarly used with other orthopaedic surgical operations and applications which would be evident to one skilled in the art. Particularly, the present invention is preferably used in conjunction with a CT-free CAS system for THR, for instance as defined in U.S. Application Ser. No. 60/415,809 filed Oct. 4, 2002, the full contents of which are incorporated herein by reference.

Further, while the bone reference member described above is preferably used in connection with the subsequent aspect of the present invention, an optically trackable CAS bone reference member as defined in U.S. application Ser. No. 10/263,711 filed Oct. 4, 2002 or U.S. application Ser. No. 10/263,708 filed Oct. 4, 2002 may alternately be employed. The above-noted references are assigned to the same assignee as the present invention, and are incorporated by reference herewith.

In a CT-free surgical procedure, a plurality of points on a surface of the bone element can then be digitized to create a digitized computer surface model of a portion of the bone element. The surface digitization is preferably done by acquiring the plurality of points, either pre-determined and sequentially identified by the CAS to the surgeon or randomly selected by the surgeon, on the surface of the bone element using a digitizer such as a calibrated CAS probe. Such landmark digitization techniques permit intra-operatively acquired surface points, preferably acquired on specific predetermined bony landmarks of the bone element surface, to be used to create a computerized anatomical reference model of the bone element.

Replacement of a natural hip with a prosthetic hip replacement can result in a change in the position of the leg of the treated hip in the medio-lateral axis of the pelvic coordinate system. A discrepancy between the final leg length of the limb on the treated hip side relative to the length of the non-treated limb can also result. Using CT-free landmark digitization techniques as defined above, the present CAS system comprises means for determining these variations in the limb positions based on pre-hip joint dislocation and post-hip joint reduction values, without requiring that the limb in question to be tracked using a bone reference member fastened thereto. The system and method preferably used to determine these limb position measurements follows.

An antero-posterior (AP) X-ray, taken pre-operatively or at least prior to the joint dislocation and subsequent joint reduction, is preferably used to determine a pre-operative, or pre-joint dislocation, value of the natural limb length discrepancy, which is the difference in length between the two limbs. In severe cases, such a length discrepancy between legs can be quite pronounced. Even in less extreme cases where bone degeneration has less severely affected the total leg length of the worn hip, and the natural leg length discrepancy is accordingly slighter, accurate measurement of the pre-operative limb length discrepancy is preferably determined. Although this is preferably done using an AP X-ray of the patient, other means of determining such a pre-operative difference between natural leg lengths can also be used.

While in a preferred embodiment the present invention is particularly described with regard to a THR, the intra-operative limb displacement required by the surgery includes a hip joint dislocation and subsequent joint reduction, once the natural hip has been replaced by the necessary prosthetic implants. However, as the present invention can be used in connection with other limb surgical procedures, the term reduction as used herein is defined to include the replacement or realignment of a body part in normal position or an initial position. Similarly, surgical limb displacement, as used herein, is intended to include hip joint dislocation and other limb movement as required for any particular orthopaedic surgical procedure. The term pre-operative, as used herein, is defined as being prior to such a joint dislocation or other surgical limb displacement. While this can be prior to the actual entire surgical procedure, in the traditional sense of the word pre-operative, it nonetheless similarly includes actions taken during the surgical procedure, but prior to the surgical limb displacement as defined.

Initially, a first digitization of a position defining landmark on the target limb to be treated is performed, prior to the joint dislocation and subsequent reduction of the hip joint being replaced, or before any other similar limb displacement required by the particular orthopaedic surgery being performed. The term pre-joint dislocation position as used herein is defined to include the position of the target limb prior to the dislocation of the natural joint. The term post-joint reduction position as used herein is defined to comprise the position of the target limb following the reduction of the artificial joint of the target limb. The position defining landmark is preferably a bony landmark chosen such that it is easily recognizable and identifiable, and is preferably located on the femur of the target leg, in the case of a total hip replacement surgery. The points chosen for the landmark digitization can be marked with an electro-surgical cutter or other bone identification means, which ensures that precise indication of the location of the digitized bony landmark is provided, such that a second post-joint reduction digitization can be performed by choosing points on the same landmark. The second digitization of the exact same landmark is performed after the installation and reduction of the artificial hip joint. It is important to ensure that the target limb, namely the treated leg, is placed in the same position with regard to the pelvis bone for the digitization of both points or surfaces. Although the position defining landmark on the target limb is preferably an actual bony landmark thereon, the term position defining landmark as defined herein is intended to include man-made landmarks, such as for example, bone reference members which are trackable by the CAS system.

The digitization of the bone surface, by a digitizer such as a calibrated probe or pointer that is identifiable by the CAS system, permits the means for determining coordinates of the CAS system to identify the position and orientation of at least the digitized surface in relation to a bone reference member fixed to another point on the body, such as the pelvic bone for example. Therefore, although the digitized limb surface is not operatively tracked, its position relative to a bone reference member on an independent bone element can thereby be determined both before and after the hip replacement. The digitized points can be projected onto the longitudinal and medio-lateral axes of the pelvic coordinate system of the patient, as defined by the first tracked bone reference member which defines the base coordinate reference system.

Although the digitization of the position defining landmarks on the un-tracked target limb preferably comprises acquiring points thereon using a CAS pointer, the process of digitization of the position defining landmarks, as defined herein, is intended to include the position identification of a trackable bone reference member by the CAS system. Accordingly, although the preferred embodiment of the present invention permits the position of an un-tracked limb to be determined, it is similarly possible to determine limb length discrepancy and limb medio-lateral offset, as will be described in further detail below, of a target limb which has a bone reference member fixed thereto, and is thus tracked by the CAS system. In this case, the first and second digitizations of the position defining landmark, which is a second trackable CAS bone reference member, comprise using the CAS system to identify the positions thereof in the base coordinate system, which is defined by the first bone reference member fixed to the pelvis of the patient. The relative positions of one trackable bone reference member to the other, determined both before joint dislocation and after the joint reduction by the first and second digitizations respectively, are therefore similarly used to determine the limb length discrepancy and limb medio-lateral offset. It therefore follows, in this alternate embodiment, that a first bone reference member that is trackable by the CAS system is fixed to a bone element that is distinct from the target limb, such as the pelvic bone, and a second trackable bone reference member is fixed to the target limb, such as the femur of the target leg. However, in this alternate embodiment in which the target limb is tracked, the additional steps of performing a digitization of the target limb coordinate system, and performing a digitization of the center of rotation of the treated joint, either on the acetabulum or the femoral head for example, are preferably performed.

The difference between the projected coordinates of the first pre-joint dislocation digitized points and the second post-joint reduction digitized points is accordingly computed by the CAS system's means for determining one of a limb length discrepancy value and a limb medialization value, in order to determine the limb length discrepancy and the operated limb medialization. Limb medio-lateral offset, or medialization, as used herein is broadly defined as the difference between the pre-joint dislocation and post-joint reduction positions of a limb along the medio-lateral axis. Limb length discrepancy as used herein is broadly defined as the difference between the length of the target limb and the length of the untreated limb.

These two measurements are computed slightly differently. The limb medio-lateral offset is calculated by determining the difference between the medio-lateral axis coordinates of the projected pre and post-operative digitized points on the treated leg. Namely:
limb medialization =−Z1+Z2,
   where Z1 and Z2 are respectively the pre and post operative coordinates of the digitized points projected on the medio-lateral axis of the pelvic coordinate system. Therefore, the limb medio-lateral offset as calculated by the CAS system determines the difference in position, in the medio-lateral axis, of the treated hip after the total hip replacement has been performed, in comparison with its position pre-operatively. This generally provides information regarding the effect of the total hip replacement on the medio-lateral positioning of the treated leg.

While the primary concern to surgeons is generally the post-THR limb medio-lateral offset for the treated leg only, it is nevertheless possible to determine the treated limb medialization relative to the non-treated leg, rather than relative to the pre-operative treated limb medio-lateral position. However, as this may require digitization of a precisely corresponding points or bone surface landmark on both the target and non-treated leg, which may require unnecessary invasiveness on the non-treated leg, post-THR limb medialization of the treated leg relative to the non-treated leg is usually not determined. However, as per the limb length discrepancy determination, a pre-operatively taken X-ray can similarly be used to determine the position of the non-treated leg so that post-THR limb medialization values of the treated leg relative to the non-treated leg can be measured without undue invasiveness.

Limb length discrepancy between the treated and non-treated legs is determined as follows by the CAS system. The pre and post-operative values of the treated leg digitized landmarks projected onto the longitudinal axis, are related to the pre-operative leg length discrepancy between the treated and non-treated leg, which is measured from the pre-operative antero-posterior X-ray. Namely:
limb length discrepancy=ΔpreopLL−Y1+Y2,
   where ΔpreopLL is the pre-operatively measured leg length discrepancy and Y1 and Y2 are respectively the coordinates of the pre and post-operative digitized points on the longitudinal axis of the pelvic coordinate system. The post-operative leg length discrepancy between the treated leg and the non-treated leg can thereby be determined by the CAS system.

The embodiments of the invention described above are intended to be exemplary only. The scope of the invention is therefore intended to be limited solely by the scope of the appended claims.

1. A surgical bone reference assembly, adapted for communication with a computer assisted surgical system, comprising:
a bone anchor member, engageable to a bone element of a patient such that substantially no relative movement therebetween is possible;
a trackable member comprising a detectable element adapted to be located and tracked in three dimensional space by the computer assisted surgical system, thereby defining position and movement of said trackable member;
an adjustable support member having said trackable member disposed at a first end thereof, a second end of said support member being removably fastenable to said bone anchor member by an attachment member, said support member permitting variable positioning of said trackable member relative to said bone anchor member and being lockable to fix said trackable member in a desired position and orientation relative to said bone anchor member; and
said support member, via said attachment member, being intra-operatively detachable from said bone anchor member and subsequently re-fastenable thereto such that said trackable member is in said desired position and orientation relative to the bone element.

2. The surgical bone reference assembly as defined in claim 1, wherein said attachment member comprises an alignment element, permitting said support member to be re-positioned relative to said bone anchor member such that said trackable member is in said desired position, and a fastener element, providing locked engagement of said support member to said bone anchor member.

3. The surgical bone reference assembly as defined in claim 2, wherein said fastener element comprises a threaded member for providing threaded engagement between said support member and said bone anchor member.

4. The surgical bone reference assembly as defined in claim 2, wherein said alignment element of said attachment member comprises mating pin and socket engagement members, provided on one and the other of said support member and said bone anchor member.

5. The surgical bone reference assembly as defined in claim 4, wherein said pin engagement member comprises an elongated base link member of said support member that is receivable within said socket engagement member comprising a central bore of said bone anchor member, said base link member and said central bore being fastenable together by a locking nut of said attachment member.

6. The surgical bone reference assembly as defined in claim 5, wherein alignment pins transversely project from said base link member for receipt in corresponding alignment grooves in said bone anchor member.

7. The surgical bone reference assembly as defined in claim 6, wherein said alignment grooves are substantially V-shaped.

8. The surgical bone reference assembly as defined in claim 1, wherein said adjustable support member is articulated, permitting selective positioning of said trackable member relative to said bone anchor member between predetermined fixed positions.

9. The surgical bone reference assembly as defined in claim 1, wherein said bone anchor member is engaged to the bone element by at least one bone mounting pin, a proximal end thereof being anchored to the bone element, the bone anchor member being fastenable to a distal end of the bone mounting pin.

10. The surgical bone reference assembly as defined in claim 9, wherein the bone anchor member comprises at least an aperture therein for receiving the distal end of the bone mounting pin, the bone anchor member being displaceable along said bone mounting pin and comprising a locking member for fixing said bone anchor member in a selected position thereon.

11. The surgical bone reference assembly as defined in claim 10, wherein said bone anchor member comprises three apertures, each adapted for receiving one of said bone mounting pins.

12. The surgical bone reference assembly as defined in claim 11, wherein said apertures are inclined relative to each other such that travel of said bone anchor member on said bone mounting pins is limited, thereby increasing stability of the bone anchor member when fixed in place to said bone mounting pins by said locking member.

13. The surgical bone reference assembly as defined in claim 1, wherein all surfaces are substantially seamless and are at least one of substantially exposed and exposable, such that said surfaces can easily be pressure cleaned and autoclaved to remove biological matter therefrom.

14. A method for monitoring position and movement of a bone element using a computer assisted surgical system comprising:
fastening a bone anchor member to the bone element;
attaching an adjustable support member to said bone anchor member, said adjustable support member having a trackable member fixed thereto, said trackable member including a detectable element being locatable and trackable in three dimensional space by said computer-assisted surgical system;
adjusting said trackable member into a desired position and orientation relative to sensing elements of said computer-assisted surgical system;
locking said adjustable support member in place such that said trackable member is fixed in said desired position and orientation relative to sensing elements of said computer-assisted surgical system;
performing a registration of the bone element;
detaching said adjustable support member from said bone anchor member; and
re-fastening said adjustable support member to said bone anchor member, said trackable member being in said desired position and orientation without requiring readjustment and said bone element being locatable and trackable using said computer assisted surgical system without requiring re-registration of said bone element.

15. The method as defined in claim 14, wherein adjusting said trackable member comprises using said computer assisted surgical system to determine said desired position and orientation of said trackable member, said desired position and orientation of said trackable member permitting substantially uninterrupted communication between said detectable elements of said trackable member and said sensing elements of said computer assisted surgical system.

16. The method as defined in claim 14, wherein fastening said bone anchor member to the bone element further comprises: anchoring a proximal end of at least one bone mounting pin to the bone element; and fastening the bone anchor member to a distal end of the at least one bone mounting pin.

17. The method as defined in claim 16, wherein the bone anchor member includes an aperture therein for receiving the bone mounting pin therein, further comprising displacing the bone anchor member along the bone mounting pin until a selected position thereon is reached, and fixing the bone anchor member to the bone mounting pin at said selected position.

18. A computed tomography (CT) free computer assisted surgery (CAS) system for determining a change in position of an un-tracked target limb undergoing orthopaedic surgery, comprising:
a bone reference member trackable by said CAS system and engaged with a bone element distinct from said target limb;
means for locating said bone reference member and determining a base coordinate system relative thereto;
a digitizer, trackable by said CAS system, for performing a first and a second digitization of a landmark on said target limb;
means for determining pre-joint dislocation coordinates in said base coordinate system from said first digitization and post-joint reduction coordinates in said base coordinate system from said second digitization, and for determining longitudinal axis components and medio-lateral axis components of said pre-joint dislocation coordinates and said post-joint reduction coordinates; and
means for determining at least one of a post-joint reduction limb length discrepancy value and a target limb medio-lateral offset value, said post-joint reduction limb length discrepancy value being computed using said longitudinal axis components and said target limb medio-lateral offset value being computed using said medio-lateral axis components.

19. The system as defined in claim 18, further comprising a means for displaying at least one of said post-joint reduction limb length discrepancy value and said target limb medio-lateral offset value.

20. The system as defined in claim 18, wherein said target limb is a leg and said landmark is a femoral bone landmark.

21. The system as defined in claim 18, wherein said digitizer is a CAS pointer.
